(19) United States
(12) Patent Application Publication
(10) Pub. No.: US 2026/0076987 A1
(43) Pub. Date: Mar. 19, 2026

(54) INJECTABLE HYDROGEL FOR PET ENDOMETRITIS, AND PREPARATION METHOD AND USE THEREOF

(71) Applicant: Tarim University, Alar City (CN)

(72) Inventors: Junfeng LIU, Alar City (CN); Pan LIU, Alar City (CN); Guang HAO, Alar City (CN); Yaofeng ZHANG, Alar City (CN)

(21) Appl. No.: 18/985,589

(22) Filed: Dec. 18, 2024

(30) Foreign Application Priority Data
Sep. 13, 2024 (CN) 202411285058.5

(51) Int. Cl. A61K 31/7034; A61K 9/00; A61K 9/06; A61K 47/12; A61K 47/24; A61K 47/36

(52) U.S. Cl. CPC A61K 31/7034; A61K 9/0019; A61K 9/06; A61K 47/12; A61K 47/24; A61K 47/36

(57) ABSTRACT

An injectable hydrogel for pet endometritis is provided, belonging to the technical field of veterinary drug preparation. The injectable hydrogel includes the following raw materials: forsythiaside A, chitosan, sodium β-glycerophosphate, sodium hyaluronate, acetic acid, and water. By combining the technical advantages of thermosensitive gel with injectable gel, forsythiaside A is used in combination with the chitosan to obtain an injectable hydrogel with excellent antibacterial properties. Encapsulating forsythiaside A with the chitosan not only enhances an antibacterial effect of forsythiaside A, but also prolongs a release time of the drug and can effectively shorten a duration required for repairing inflammatory damages, thereby reducing the probability of secondary infection in the uterine cavity.

### CROSS-REFERENCE TO RELATED APPLICATION

This patent application claims the benefit and priority of Chinese Patent Application No. 2024112850585 filed with the China National Intellectual Property Administration on Sep. 13, 2024, the disclosure of which is incorporated by reference herein in its entirety as part of the present application.

### TECHNICAL FIELD

The present disclosure relates to the technical field of veterinary drug preparation, and in particular to an injectable hydrogel for pet endometritis, its preparation method, and use thereof.

### BACKGROUND

Endometritis, characterized by severe inflammatory reactions endometrial and myometrial layers, is one of the primary diseases leading to decreased reproductive performance in female livestock. Endometritis has a complex etiology, and infections by pathogenic microorganisms are one of the main causes, among which Escherichia coli and Staphylococcus aureus are the main pathogens causing endometritis. The invasion of pathogenic microorganisms into the endometrial and myometrial layers triggers inflammatory reactions, resulting in endometrium damages and leading to reproductive disorders.

Currently, the local or systemic administration of antibiotics is a common clinical treatment for endometritis, yet it presents issues such as the propensity for drug residues to develop, the presence of high drug residues, and the occurrence of severe toxic side effects. For example, Chinese patent application CN117717520A has disclosed a preparation method of a novel uterine injection. The uterine injection uses a cephalosporin antibiotic, ceftiofur crystalline free acid (CCFA), as a main drug ingredient, and specifically includes the following raw materials: 5% to 10% of CCFA, 5% to 10% of emulsifier, 5% to 10% of oil, 6% to 10% of calcium silicate, 3% to 5% of hydrophilic silica, 5% to 10% of modified sesbania powder, 2% to 5% of surfactant, 1% to 3% of antioxidant, and 37% to 68% of injection matrix.

Therefore, it is urgent to develop novel drug preparations to replace antibiotics in the treatment of endometritis. Plant-derived drugs are not easy to develop drug resistance, have low drug residues, and generate little toxic side effects. The active ingredient of the plant-derived drugs, forsythiaside A, has antibacterial and anti-inflammatory pharmacological effects, but is still not widely used in the treatment of veterinary clinical diseases due to the restrictions of administration methods, preparation processes, and drug utilization.

The dried fruit of Fructus Forsythiae, used as a medicinal part, demonstrates effect of clearing away heat and detoxifying, and has been widely used in the treatment of diseases such as acute nephritis, erysipelas, and ulcers. Moreover, the Fructus Forsythiae has a wide range of pharmacological effects. Forsythiaside A, a phenylethanol glycoside compound extracted from the dried fruit of Fructus Forsythiae, is one of the main components of Fructus Forsythiae, and has antibacterial, antioxidant, and antiviral effects. Many studies have shown that forsythiaside A, as one of the strongest antibacterial ingredients in Fructus Forsythiae, can effectively inhibit Staphylococcus aureus to achieve anti-inflammatory and anti-infection effects.

Injectable hydrogels possess characteristics such as biocompatibility, high permeability, and low invasiveness, thereby offering superior benefits over traditional hydrogels. For example, using injectable hydrogels does not require complex surgical procedures. The injectable hydrogels can ultimately reduce discomfort and treatment costs for sick pets through a simple and minimally invasive procedure.

The current challenge is to develop an injectable hydrogel that utilizes forsythiaside A, an active ingredient from plant-derived drugs, as a raw material.

### SUMMARY

In order to solve the above problems, an objective of the present disclosure is to provide an injectable hydrogel for pet endometritis. In the present disclosure, forsythiaside A is encapsulated with chitosan, and antibacterial activity of the chitosan is combined to enhance antibacterial properties of forsythiaside A. In this way, an injectable hydrogel is prepared, which has excellent antibacterial properties, can reduce intrauterine infection, effectively shorten a repair time of inflammatory damages, and is convenient for vaginal administration.

To achieve the objective of the present disclosure, the following technical solutions are proposed.

The present disclosure provides an injectable hydrogel for pet endometritis, including the following raw materials in parts by mass:

0.1 parts to 2 parts of forsythiaside A, 0.8 parts to 1.4 parts of chitosan, 9.5 parts to 15 parts of sodium β-glycerophosphate, 0.19 parts to 0.3 parts of sodium hyaluronate, 0.1 parts to 10 parts of acetic acid, and 50 parts to 100 parts of water.

Additionally, the injectable hydrogel for pet endometritis comprises the following components in parts by mass: 0.8 parts of forsythiaside A, 1.12 parts of chitosan, 14.4 parts of sodium β-glycerophosphate, 0.192 parts of sodium hyaluronate, 0.3 parts of acetic acid, and 81 parts of water.

Another objective of the present disclosure is to provide a preparation method of the injectable hydrogel for pet endometritis, including the following steps:
(1) weighing the raw materials according to the parts by mass;
(2) adding the chitosan into one part of the water, and then adding acetic acid to allow dissolution by a resulting mixture stirring to obtain solution 1;
(3) adding forsythiaside A into one part of water, and then stirring evenly to allow ultrasonic co-dissolution to obtain solution 2;
(4) adding sodium β-glycerophosphate and sodium hyaluronate into remaining parts of water, and then stirring evenly to obtain solution 3;
(5) adding solution 2 dropwise into solution 1, and then stirring evenly to obtain solution 4; and
(6) adding solution 3 dropwise into solution 4, and then stirring evenly to obtain the injectable hydrogel for pet endometritis.

Further, the chitosan and the one part of water in step (2) are at a mass ratio of (0.02-0.03):1.

Further, forsythiaside A and the one part of water in step (3) are at a mass ratio of (0.8-0.9):1.

Further, solution 1 and solution 3 are at a volume ratio of (6-8):(2-4).

Further, the adding dropwise in step (5) is conducted at a room temperature or in an ice-water bath.

Further, solution 3 and solution 4 are allowed to stand at 0° C. to 4° C. for 30 min to 40 min before the dropwise addition in step (6) is conducted.

Further, the dropwise addition in step (6) is conducted in an ice-water bath.

Further, the dropwise addition in step (6) is conducted under stirring at 1,000 r/min to 1,200 r/min.

A third objective of the present disclosure is to provide use of the injectable hydrogel for pet endometritis in preparation of an antibacterial and anti-inflammatory drug.

Compared with the prior art, the present disclosure has the following beneficial effects:

The present disclosure provides an injectable hydrogel for pet endometritis. By combining the technical advantages of thermosensitive and injectable gels, forsythiaside A is used in combination with chitosan to obtain an injectable hydrogel with excellent antibacterial properties. In particular, encapsulating forsythiaside A with the chitosan enhances antibacterial effect, thereby reducing the probability of secondary infection in the uterine cavity.

### BRIEF DESCRIPTION OF THE DRAWINGS

FIG. 1 shows appearance changes of the injectable hydrogel for pet endometritis prepared in Example 1 of the present disclosure;

FIG. 2 shows a scanning electron microscopy (SEM) image of the injectable hydrogel for pet endometritis prepared in Example 1 of the present disclosure;

FIG. 3 shows a particle size of the injectable hydrogel for pet endometritis prepared in Example 1 of the present disclosure;

FIG. 4 shows a Zeta potential class of the injectable hydrogel for pet endometritis prepared in Example 1 of the present disclosure;

FIG. 5 shows an antibacterial effect of the injectable hydrogel for pet endometritis prepared in Example 1 of the present disclosure; and

FIG. 6A-FIG. 6D show results of H&E staining of mouse uterine tissues in different treatment groups.

### DETAILED DESCRIPTION OF THE EMBODIMENTS

The present disclosure is further described below in conjunction with examples, and the “parts” described in the following examples are all “parts by mass”.

### Example 1

An injectable hydrogel for pet endometritis included the following raw materials:

0.8 parts of forsythiaside A (bulk drug), 1.12 parts of chitosan, 14.4 parts of sodium β-glycerophosphate, 0.192 parts of sodium hyaluronate, 0.3 parts of acetic acid, and 81 parts of water.

A preparation method of the injectable hydrogel for pet endometritis included the following steps:
(1) the raw materials were weighed according to the parts by mass;
(2) the chitosan was added into 56 parts of water, and then acetic acid was added to allow dissolution by stirring to obtain solution 1;
(3) forsythiaside A was added into 1 part of the water, and then stirred evenly to allow ultrasonic co-dissolution to obtain solution 2;
(4) sodium β-glycerophosphate and sodium hyaluronate were added into the remaining water, and then stirred evenly to obtain solution 3 (where the chitosan solution and the sodium β-glycerophosphate/sodium hyaluronate solution were at a volume ratio of 7:3);
(5) solution 2 was added dropwise into solution 1, and then stirred evenly to obtain solution 4; and
(6) solution 3 and solution 4 were allowed to stand in an ice-water bath for 40 min, solution 3 was added dropwise to solution 4, and then stirred evenly to obtain the injectable hydrogel for pet endometritis. The composite gel included a composite of forsythiaside A and chitosan.

### Example 2

An injectable hydrogel for pet endometritis included the following raw materials:

0.8 parts of forsythiaside A (bulk drug), 1.28 parts of chitosan, 9.6 parts of sodium β-glycerophosphate, 0.128 parts of sodium hyaluronate, 0.3 parts of acetic acid, and 81 parts of water.

A preparation method of the injectable hydrogel for pet endometritis included the following steps:
(1) the raw materials were weighed according to the parts by mass;
(2) the chitosan was added into 64 parts of water, and then acetic acid was added to allow dissolution by stirring a resulting mixture to obtain solution 1;
(3) forsythiaside A was added into 1 part of water, and then stirred evenly to allow ultrasonic co-dissolution to obtain solution 2;
(4) sodium β-glycerophosphate and sodium hyaluronate were added into the remaining water, and then stirred evenly to obtain solution 3 (where the chitosan solution and the sodium β-glycerophosphate/sodium hyaluronate solution were at a volume ratio of 8:2);
(5) solution 2 was added dropwise into solution 1, and then stirred evenly to obtain solution 4; and
(6) solution 3 and solution 4 were allowed to stand in an ice-water bath for 40 min, solution 3 was added dropwise to solution 4, and then stirred evenly to obtain the injectable hydrogel for pet endometritis. The composite gel included a composite of forsythiaside A and chitosan.

### Example 3

An injectable hydrogel for pet endometritis included the following raw materials:

0.8 parts of forsythiaside A (bulk drug), 0.96 parts of chitosan, 19.2 parts of sodium β-glycerophosphate, 0.192 parts of sodium hyaluronate, 0.3 parts of acetic acid, and 81 parts of water.

A preparation method of the injectable hydrogel for pet endometritis included the following steps:
(1) the raw materials were weighed according to the parts by mass;
(2) the chitosan was added into 48 parts of water, and then acetic acid was added to allow dissolution by stirring to obtain solution 1;
(3) forsythiaside A was added into 1 part of water, and then stirred evenly to allow ultrasonic co-dissolution to obtain solution 2;
(4) sodium β-glycerophosphate and sodium hyaluronate were added into the remaining water, and then stirred evenly to obtain solution 3 (where the chitosan solution and sodium β-glycerophosphate/sodium hyaluronate solution were at a volume ratio of 6:4);
(5) solution 2 was added dropwise into solution 1, and then stirred evenly to obtain solution 4; and
(6) solution 3 and solution 4 were allowed to stand in an ice-water bath for 40 min, solution 3 was added dropwise to solution 4, and then stirred evenly to obtain the injectable hydrogel for pet endometritis. The composite gel included a composite of forsythiaside A and chitosan.

A thermosensitivity of the injectable hydrogels prepared in Examples 1 to 3 was tested by an “inversion method”. Three milliliters of the prepared injectable hydrogel was added into a glass bottle and placed in a 37° C. constant-temperature water bath while timing and observing fluidity of a hydrogel liquid. When the fluidity became low, the glass bottle was taken out every 30 s and inverted 180°. If the hydrogel liquid sample did not flow for 30 s, it indicated that the hydrogel liquid had turned into hydrogel, and a time of duration for gelation was recorded at this time. Results are shown in Table 1.

TABLE 1

| | Factor | | | | |
|---|---|---|---|---|---|
| | | | | V (chitosan):V | |
| | | | | (sodium | |
| | | Sodium | Sodium | β-glycero- | |
| | Chito- | β-glycero- | hyaluro- | phosphate/sodium | Gelation |
| Example | san | phosphate | nate | hyaluronate) | time/s |
| Example 1 | 1.12 | 14.4 | 0.192 | 7:3 | 272.23 ± |
| | | | | | 2.045 |
| Example 2 | 1.28 | 9.6 | 0.128 | 8:2 | 287.00 ± |
| | | | | | 1.433 |
| Example 3 | 0.96 | 19.2 | 0.256 | 6:4 | 307.57 ± |
| | | | | | 2.035 |

### Comparative Example 1

An injectable hydrogel for pet endometritis included the following raw materials:

0.8 parts of forsythiaside A (bulk drug), 1.12 parts of chitosan, 14.4 parts of sodium β-glycerophosphate, 0.3 parts of acetic acid, and 81 parts of water.

A preparation method of the injectable hydrogel for pet endometritis included the following steps:
(1) the raw materials were weighed according to the parts by mass;
(2) the chitosan was added into 56 parts of water, and then acetic acid was added to allow dissolution by stirring to obtain solution 1;
(3) forsythiaside A was added into 1 part of water, and then stirred evenly to allow ultrasonic co-dissolution to obtain solution 2;
(4) sodium β-glycerophosphate was added into the remaining water, and then stirred evenly to obtain solution 3 (where the chitosan solution and the sodium β-glycerophosphate solution were at a volume ratio of 7:3);
(5) solution 2 was added dropwise into solution 1, and then stirred evenly to obtain solution 4; and
(6) solution 3 and solution 4 were allowed to stand in an ice-water bath for 40 min, solution 3 was added dropwise to solution 4, and then stirred evenly to obtain the injectable hydrogel for pet endometritis. The composite gel included a composite of forsythiaside A and chitosan.

### Comparative Example 2

An injectable hydrogel for pet endometritis included the following raw materials:

0.8 parts of forsythiaside A (bulk drug), 1.28 parts of chitosan, 9.6 parts of sodium β-glycerophosphate, 0.3 parts of acetic acid, and 81 parts of water.

A preparation method of the injectable hydrogel for pet endometritis included the following steps:
(1) the raw materials were weighed according to the parts by mass;
(2) the chitosan was added into 64 parts of water, and then acetic acid was added to allow dissolution by stirring to obtain solution 1;
(3) forsythiaside A was added into 1 part of water, and then stirred evenly to allow ultrasonic co-dissolution to obtain solution 2;
(4) sodium β-glycerophosphate was added into the remaining water, and then stirred evenly to obtain solution 3 (where the chitosan solution and the sodium β-glycerophosphate solution were at a volume ratio of 8:2);
(5) solution 2 was added dropwise into solution 1, and then stirred evenly to obtain solution 4; and
(6) solution 3 and solution 4 were allowed to stand in an ice-water bath for 40 min, solution 3 was added dropwise to solution 4, and then stirred evenly to obtain the injectable hydrogel for pet endometritis. The composite gel included a composite of forsythiaside A and chitosan.

### Comparative Example 3

An injectable hydrogel for pet endometritis included the following raw materials:

0.8 parts of forsythiaside A (bulk drug), 0.96 parts of chitosan, 19.2 parts of sodium β-glycerophosphate, 0.3 parts of acetic acid, and 81 parts of water.

A preparation method of the injectable hydrogel for pet endometritis included the following steps:
(1) the raw materials were weighed according to the parts by mass;
(2) the chitosan was added into 48 parts of water, and then acetic acid was added to allow dissolution by stirring to obtain solution 1;
(3) forsythiaside A was added into 1 part of water, and then stirred evenly to allow ultrasonic co-dissolution to obtain solution 2;
(4) sodium β-glycerophosphate was added into the remaining water, and then stirred evenly to obtain solution 3 (where the chitosan solution and the sodium β-glycerophosphate solution were at a volume ratio of 6:4);
(5) solution 2 was added dropwise into solution 1, and then stirred evenly to obtain solution 4; and
(6) solution 3 and solution 4 were allowed to stand in an ice-water bath for 40 min, solution 3 was added dropwise to solution 4, and then stirred evenly to obtain the injectable hydrogel for pet endometritis. The composite gel included a composite of forsythiaside A and chitosan.

A thermosensitivity of the injectable hydrogels prepared in the comparative examples was tested by the “inversion method”. Three milliliters of the prepared injectable hydrogel was added into a glass bottle and placed in a 37° C. constant-temperature water bath while timing and observing fluidity of a hydrogel liquid. When the fluidity became low, the glass bottle was taken out every 30 s and inverted 180°. If the hydrogel liquid sample did not flow for 30 s, it indicated that the hydrogel liquid had turned into hydrogel, and a time of duration for gelation was recorded at this time. Results are shown in Table 2.

TABLE 2

| | Factor | | | | |
|---|---|---|---|---|---|
| | | | | V (chitosan):V | |
| | | Sodium | Sodium | (sodium | |
| | Chito- | β-glycero- | hyaluro- | β-glycero- | Gelation |
| Example | san | phosphate | nate | phosphate) | time/s |
| Comparative | 1.12 | 14.4 | 0 | 7:3 | 313.00 ± |
| Example 1 | | | | | 1.414 |
| Comparative | 1.28 | 9.6 | 0 | 8:2 | 308.33 ± |
| Example 2 | | | | | 1.700 |
| Comparative | 0.96 | 19.2 | 0 | 6:4 | 310.33 ± |
| Example 3 | | | | | 1.247 |

The hydrogel prepared in Example 1 was subjected to the following characterizations and experiments.

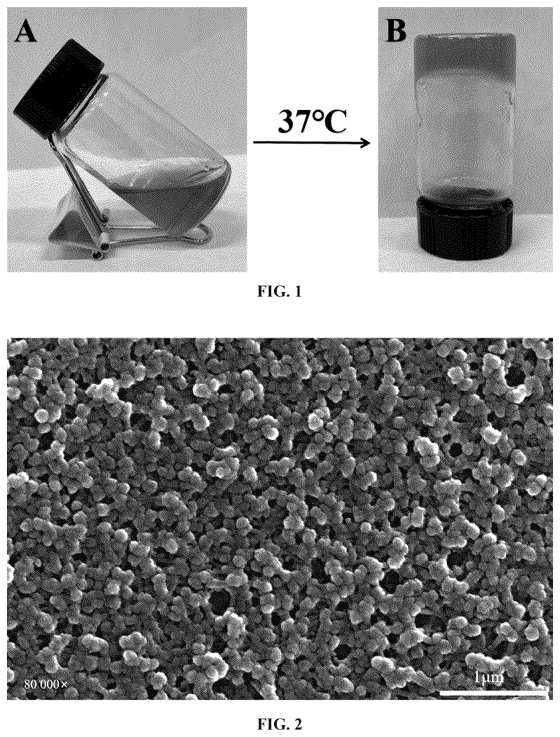

(I) A schematic diagram for appearance changes of the hydrogel prepared in Example 1 at different temperatures is shown in FIG. 1. As shown in FIG. 1, the hydrogel prepared in Example 1 was a brown-yellow sol-like substance with uniform transparency, no drug precipitates were observed, and it could be transformed into a brown-yellow solid gel at body temperature (37° C.). This proved that the hydrogel of the present disclosure could quickly form a gel to cover the affected area after being injected into the pet's uterus to exert an effect.

(II) The hydrogel sample prepared in Example 1 was freeze-dried and then adhered to a SEM sample plate with conductive glue. Gold was sputtered using an ion sputtering device, and then a structure of the sample was observed by SEM. The SEM results are shown in FIG. 2. As shown in FIG. 2, the hydrogel prepared in Example 1 formed a three-dimensional grid pore structure with uniform pore size, which could carry drugs well and facilitate the absorption and release of nutrients and metabolites.

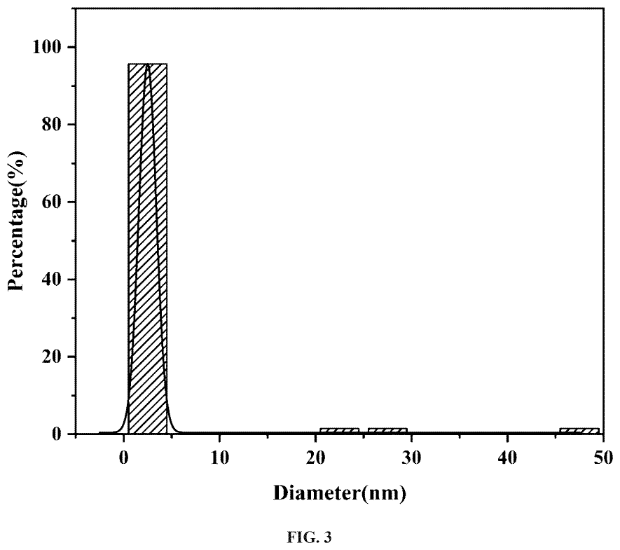

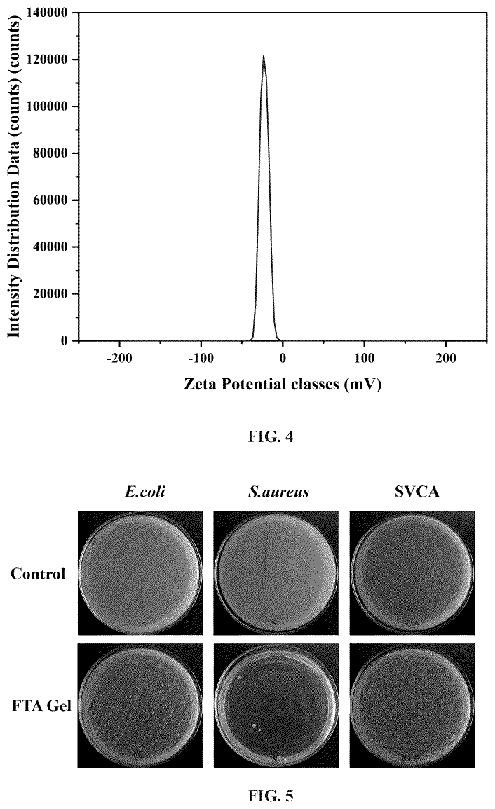

(III) 1 mL of hydrogel prepared in Example 1 was diluted 100 times with deionized water, sonicated for 30 min, and stored for later use. The particle size and Zeta potential were measured using a nanoparticle size analyzer and Zeta potential analyzer (by DLS). The particle size measurement results are shown in FIG. 3, and the Zeta potential measurement results are shown in FIG. 4.

As shown in FIG. 3 and FIG. 4, the hydrogel prepared in Example 1 had a particle size of 4.248 nm (FIG. 3). Due to the small particle size of the hydrogel, the drug could be better delivered to the medication site through the tissue space, thereby achieving the antibacterial effect and promoting tissue repair. The hydrogel had a Zeta potential of −22.5 mV (FIG. 4), indicating that the surface charge was neutral and could be adsorbed on the bacterial surface to inhibit bacterial growth.

### (IV) Observation of Antibacterial Effect by Plate Coating Method

The hydrogel prepared in Example 1 was formulated into a hydrogel solution with a drug loading concentration of 10 mg/mL. The constant dilution method was slightly modified. 0.1 g of freeze-dried hydrogel powder prepared in Example 1 was weighed and added into a shaking bottle containing 5 mL of autoclaved MH broth, and 1 mL of E. coli, S. aureus, and SCVA (1×10⁸ CFU/mL) was added. A resulting mixture was cultured at 37° C. in a constant-temperature shaker at 200 r/min for 18 h to 24 h. An obtained bacterial suspension (100.0 μL) was extracted onto the plate, the coating was diluted by 10-fold dilution method, and the diluted coating solution (100.0 μL) was added into a nutrient agar medium. The plate was further cultured at 37° C. for 24 h, and the antibacterial effect diagram is shown in FIG. 5.

As shown in FIG. 5, the results of the plate coating of the injectable gel for endometrial inflammatory lesions in pets showed a large number of colonies growing on the single bacterium E. coli, and there was a poor antibacterial effect; the results of the plate coating of the single bacterium S. aureus showed a small number of colonies growing, indicating an antibacterial effect; the results of the plate coating of the single bacterium SCVA showed a large number of colonies growing, and there was a poor antibacterial effect.

### (V) Influence of the Injectable Hydrogel for Pet Endometritis on Repair of Endometrial Inflammatory Lesions in Mice

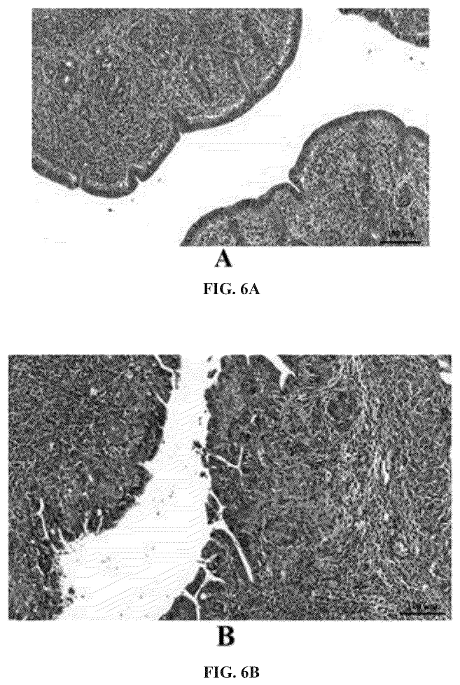

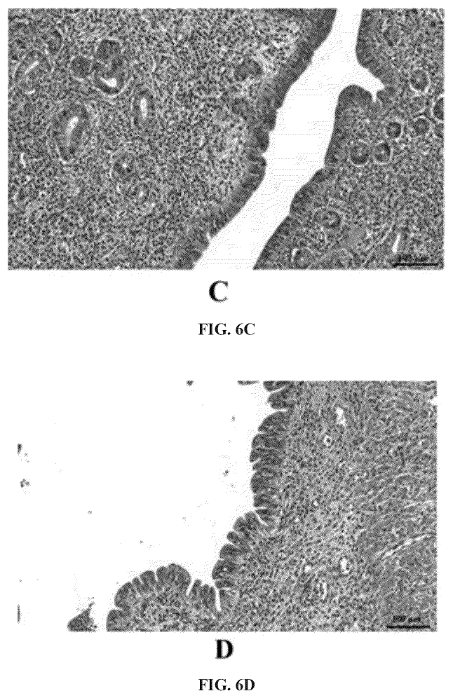

36 healthy female Kunming mice (18 g to 22 g) were injected with 0.2 mL of 1 mg/mL LPS into their uterus through vulva using a homemade uterine perfusion device to obtain a mouse endometrial inflammatory injury model. After 12 h, the mice showed depression, rough fur, and yellow secretions in the genitals, indicating that the model was successfully established. The mice were randomly divided into 4 groups (A. blank control group, B. LPS modeling group, C. injectable hydrogel for pet endometritis treatment group, and D. Forsythiaside A raw material treatment group) by a random drawing lots method, with 9 mice in each group. Group A was not treated in any way, while the other 3 groups were modeled. After modeling, the C and D groups were intrauterine-administered with forsythiaside A raw material solution and the injectable hydrogel for pet endometritis, respectively, once every 24 h, for a total of 4 times. 24 h after the last administration, 3 mice were randomly selected from each group and anesthetized and then sacrificed. Their uterine tissues were removed and flattened on sterile filter paper. The blood on the tissue was cleaned and the tissue was fixated with 4% paraformaldehyde. H&E staining was conducted to obtain H&E staining results of the uterine tissue of mice in different treatment groups, as shown in FIG. 6A-FIG. 6D.

As shown in FIG. 6A-FIG. 6D, the results of histopathological analysis suggested that the uterine tissue structure of mice in the blank control group was normal, no inflammatory infiltration was found, the endometrial cells were closely arranged, and the glands were evenly distributed. The uterus of mice in the LPS modeling group showed obvious inflammatory cell infiltration, severe shedding of endometrial cells, and destroyed glandular structure. After treatment, the pathological changes in the mouse uterine tissue of the forsythiaside A raw material treatment group were significantly reduced, but a small amount of inflammatory cell infiltration and endometrial cell shedding were still visible. After treatment, there was almost no obvious pathological change in the mouse uterine tissue of the injectable hydrogel for pet endometritis treatment group.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. An injectable hydrogel for pet endometritis, comprising the following raw materials in parts by mass:
0.1 parts to 2 parts of forsythiaside A, 0.8 parts to 1.4 parts of chitosan, 9.5 parts to 15 parts of sodium β-glycerophosphate, 0.19 parts to 0.3 parts of sodium hyaluronate, 0.1 parts to 10 parts of acetic acid, and 50 parts to 100 parts of water.

2. The injectable hydrogel for pet endometritis according to claim 1, comprising the following raw materials in parts by mass: 0.8 parts of forsythiaside A, 1.12 parts of chitosan, 14.4 parts of sodium β-glycerophosphate, 0.192 parts of sodium hyaluronate, 0.3 parts of acetic acid, and 81 parts of water.

3. A method for preparing the injectable hydrogel for pet endometritis according to claim 1, comprising the following steps:
(1) weighing the raw materials according to the parts by mass;
(2) adding the chitosan into one part of water, and then adding acetic acid to allow dissolution by stirring to obtain solution 1;
(3) adding forsythiaside A into one part of water, and then stirring evenly to allow ultrasonic co-dissolution to obtain solution 2;
(4) adding sodium β-glycerophosphate and sodium hyaluronate into remaining parts of water, and then stirring evenly to obtain solution 3;
(5) adding solution 2 dropwise into solution 1, and then stirring evenly to obtain solution 4; and
(6) adding solution 3 dropwise into solution 4, and then stirring evenly to obtain the injectable hydrogel for pet endometritis.

4. The method according to claim 3, wherein the chitosan and the one part of water in step (2) are at a mass ratio of (0.02-0.03):1.

5. The method according to claim 3, wherein forsythiaside A and the one part of water in step (3) are at a mass ratio of (0.8-0.9):1.

6. The method according to claim 3, wherein solution 1 and solution 3 are at a volume ratio of (6-8):(2-4).

7. The method according to claim 3, wherein solution 3 and solution 4 are allowed to stand at 0° C. to 4° C. for 30 min to 40 min before the adding dropwise in step (6) is conducted.

8. The method according to claim 3, wherein the adding dropwise in step (6) is conducted in an ice-water bath.

9. The method according to claim 3, wherein the adding dropwise in step (6) is conducted under stirring at 1,000 r/min to 1,200 r/min.

10. A method for treating pet endometritis, comprising administering an antibacterial and anti-inflammatory drug to the pet, wherein the antibacterial and anti-inflammatory drug comprising the injectable hydrogel according to claim 1.

11. A method for treating pet endometritis, comprising administering an antibacterial and anti-inflammatory drug to the pet, wherein the antibacterial and anti-inflammatory drug comprising the injectable hydrogel according to claim 2.
